(19) United States
(12) Patent Application Publication
(10) Pub. No.: US 2010/0003667 A1
(43) Pub. Date: Jan. 7, 2010

(54) MULTIPLE LABELLING FOR ANALYTE DETECTION

(75) Inventor: Edward Blair, Glasgow (GB)

Correspondence Address: KNOBBE MARTENS OLSON & BEAR LLP, 2040 MAIN STREET, FOURTEENTH FLOOR, IRVINE, CA, 92614, US

(73) Assignee: ITI Scotland Limited, Glasgow , (GB)

(21) Appl. No.: 12/305,947

(22) PCT Filed: Jun. 28, 2007
(86) PCT No.: PCT/GB2007/002404

§ 371 Date: Mar. 16, 2009

(30) Foreign Application Priority Data
Jun. 28, 2006 (GB) 0612825.0

(51) Int. Cl. C12Q 1/70; G01N 33/554; G01N 33/00

(52) U.S. Cl. 435/5; 435/7.32; 436/86

(57) ABSTRACT

Provided is use of a label in a method for detecting an analyte in a sample, for increasing the sensitivity of detection of the analyte, wherein, the analyte comprises a repeating protein unit, and wherein a plurality of label entities are capable of binding to a single analyte entity such that a signal obtained for an analyte from a plurality of labels is stronger than the signal obtained for an analyte with a single label.

The present invention is concerned with improved methods of detecting analytes, in particular methods that are capable of detecting viral analytes, such as whole viruses, viral particles and viral components. The method is particularly advantageous, since it is capable of detecting analytes without resorting to complex and costly amplification techniques, such as PCR.

It is a well known method to detect pathogens, such as viruses, by reacting a component of the pathogen that is characteristic of the pathogen with a label. The label may then be detected in order to determine the presence, or absence of the pathogen. In some methods, the quantity of the label may provide further information about the quantity of the pathogen. Examples of this approach include detection of nucleic acid using nucleic acid probes that are complementary to specific genes or sequences in the pathogen. Typical methods also involve the use of antibodies capable of binding to particular antigenic regions of the pathogen (such as surface proteins in a virus) to identify the specific pathogen.

In some methods, a plurality of analyte pathogens can be detected in the same procedure, by using multiplexing techniques. These techniques typically involve the use of two or more different labels which are designed to be specific for two or more specific pathogens. These labels will be individually identifiable (e.g. by possessing different fluorescent groups) so that they can be related to the different analytes even if they are present in the same reaction zone.

However, all of these methods have a significant problem in that they suffer from a lack of sensitivity, particularly if the pathogens are present in low concentration. In the past, attempts have been made to solve this problem by concentrating the sample so as to increase the available quantity of pathogen, per unit volume of sample, or (in the case of nucleic acid in particular) by amplifying the pathogen component in the sample. Although both of these techniques can lead to greater sensitivity of detection, they have the significant drawback that they greatly increase the complexity of the method. Such a method requires much more sophisticated sample preparation, requires many more reagents and needs further reaction or sample preparation zones in the apparatus. There is also a requirement to remove waste from the concentrating and/or amplification procedure. All of this increases the cost of the method and the apparatus used to carry out the method. Furthermore, although sensitivity is increased, the extra method steps introduce the possibility that additional systemic errors exist in the final data, which in turn will require additional data processing to eliminate, further increasing complexity and cost.

Accordingly, there is a requirement for a more simple and cost effective solution to increasing the sensitivity of diagnostic methods and pathogen detection methods.

It is an aim of the present invention to solve the problems associated with the known methods described above. In particular, it is an aim of the present invention to provide uses and methods of detection of analytes, and methods of diagnosis of target analytes or pathogens, which have good sensitivity, but are more simple and cost effective than known sensitive methods.

Accordingly, the present invention provides use of a label in a method for detecting an analyte in a sample, for increasing the sensitivity of detection of the analyte, wherein, the analyte comprises a repeating protein unit, and wherein a plurality of label entities are capable of binding to a single analyte entity such that a signal obtained for an analyte from a plurality of labels is stronger than the signal obtained for an analyte with a single label.

Typically the method in which the label is used comprises:
(a) contacting the sample with the label;
(b) detecting the presence and/or quantity of the label bound to the analyte; and
(c) thereby detecting the presence and/or quantity of the analyte in the sample.

The present use has the advantage that a single entity of the analyte binds to more than one label entity. Thus, a single analyte entity will give rise to a much stronger signal (e.g. a higher amplitude of emission if fluorescence detection is employed) and sensitivity is increased. In the case of a whole virus, the protein coat is largely comprised of repeating geometric structures based on several n-fold (3-, 4-, 5-, 6-fold) axes of symmetry. Multiple labelling entities can attach to the protein capsid (up to several hundred without steric hindrance in some cases) and many times the normal sensitivity can therefore be achieved. The ability of a plurality of similar or identical entities to bind to a viral protein coat (capsid) has been verified by modelling in the human rhinovirus system (Steindl T M, Crump C E, Hayden F G, Langer T, J. Med. Chem. 2005, Oct. 6; 48(20), pp 6250-60 “Pharmacophore modelling, docking, and principal component analysis based clustering: combined computer-assisted approaches to identify new inhibitors of the human rhinovirus coat protein”. Published international patent application WO 03/068222 discloses methods of reducing rhinovirus contagion and related compositions. Ryan J et al disclose a new oral rhinovirus inhibitor BTA798 in Antiviral Research 2005, 65(3) (Abs LB 11). However, these disclosures deal principally with antiviral methods for treatment of disease. The ability of certain species to bind in the above-mentioned way has never before been utilised to improve sensitivity in detection and diagnosis methods.

The present invention further provides a method for detecting an analyte in a sample, which method comprises:
(a) contacting the sample with a label capable of binding to the analyte;
(b) detecting the presence and/or quantity of the label bound to the analyte; and
(c) thereby detecting the presence and/or quantity of the analyte in the sample;
   wherein, the analyte comprises a repeating protein unit, and wherein a plurality of label entities are capable of binding to a single analyte entity such that a signal obtained for an analyte from a plurality of labels is stronger than the signal obtained for an analyte with a single label.

Preferably, the analyte comprises a repeating protein unit of a virus selected from adenoviruses, herpesviruses, rotaviruses, reoviruses, picomaviruses, papillomaviruses, polyomaviruses, parvoviruses, parvoviruses, influenza viruses, flaviviruses and retroviruses.

Also provided by the present invention is a method of diagnosing the presence of a pathogen in a subject, which method comprises:
(a) obtaining a sample from the subject;
(b) detecting the absence or the presence and/or quantity of a pathogen protein in the sample according to a method as defined in any of claims 1-11; and
(c) making a diagnosis of the subject based on the absence or the presence and/or quantity of the pathogen.

The invention will be described in more detail in the following, by way of example only, with reference to the following Figures, in which:

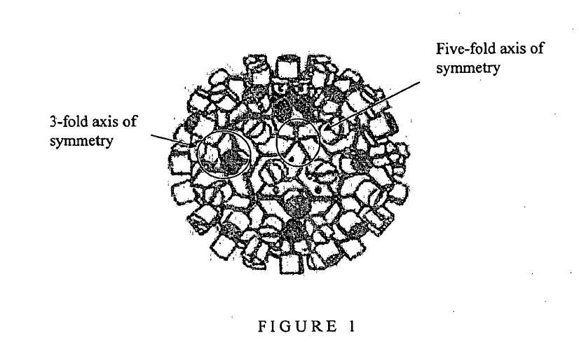

FIG. 1 shows a symmetrical repeating unit of a typical virus particle;

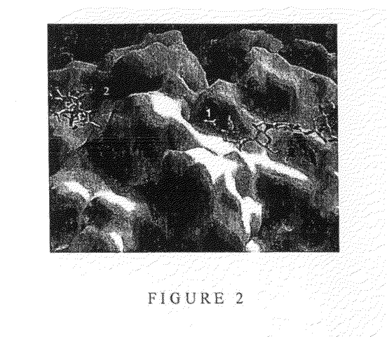

FIG. 2 shows the binding of a small molecule entity to the surface of a virus particle; and

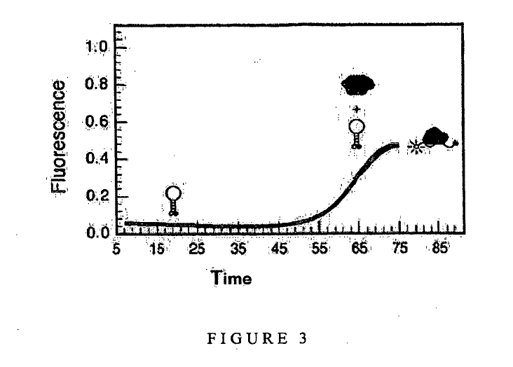

FIG. 3 shows a molecular beacon binding to a virus particle and emissions detected from it.

In the present invention, the analyte comprises a repeating protein unit. The repeating unit is required in order that more than one (preferably many) label entities may bind to the analyte. However, the type of protein is not especially limited provided that there are repeating units. Thus, the analyte may comprise a protein selected from a virus protein, a bacterial protein, and a mammalian protein (such as a human protein).

In some preferred embodiments of the present invention, the analyte may comprise a virus protein. In these embodiments, the analyte may comprise the whole of, or a portion of, a viral surface protein. However, the analyte is not otherwise especially limited, provided that a plurality of label entities are capable of binding to it. Thus, the analyte may be selected from, whole virus, viral particles, sub-viral particles, immature viral particles, and proto-viral complexes. The virus may be any type of virus, such as a bacteriophage.

The label itself is not especially limited, provided that a plurality of labelling entities can attach to a single analyte entity. The type of label employed may be selected depending on the analyte under investigation. Thus, for a whole virus, a larger label might be employed than would be for a single viral coat protein molecule, due to steric constraints. The label may comprise an antibody, a peptide, an aptamer, a nucleic acid, or an organic compound or molecule. In one embodiment, it is particularly preferred that the label comprises a capsid function inhibitor. In this embodiment, the label more preferably comprises an oxadiazole compound. The most preferred such compound is Pleconaril or a Pleconaril derivative.

Typically the label comprises a reporter group, such as a fluorescent group that is excited at a wavelength differing from its emission wavelength, Preferably, the reporter group is altered when the label binds to the analyte, as shown in FIG. 2, where the unbound entity 2 undergoes a conformational change to give the extended form of entity 1. In some preferred embodiments, the reporter group comprises. a fluorescent group that fluoresces differently in its extended form. In these embodiments, typically the label is internally quenched such that binding to an analyte (such as a virus particle) ablates quenching and leads to detectable signal output.

Preferably the pathogen is selected from a virus and a bacterium. The invention may apply to any virus. Preferred viruses include adenoviruses, herpesviruses, rotaviruses, reoviruses, picomaviruses, papillomaviruses, polyomaviruses, parvoviruses, parvoviruses, influenza viruses, flaviviruses (such as HCV) and retroviruses (such as HIV). Also included are animal viruses, plant viruses and bacterial viruses (bacteriophages), including Lambda, PhiX174, PRD1, MS2, and T-even.

In the most preferred embodiments, the virus is selected from an HCV, HIV, herpes simplex virus, human rhinovirus, influenza virus, and a vital pathogen of a non-human species.

In this method the subject is typically a mammal, and is preferably human.

As has been mentioned briefly above, it is a preferred aspect of the present invention that co-operative binding is employed in putting the invention into effect, in particular so as to make the most of the signal enhancement achieved with the present uses and methods. Such binding is typically termed ‘molecular beacon’ technology.

There are two aspects to molecular beacons, both of which are applicable to the present invention. Firstly, cooperative binding and secondly, cooperative signal amplification.

Co-operative binding is often seen in nature where the binding of one ligand promotes the binding of another to the same target macromolecule. The best known example is oxygen saturation of haemoglobin which has a “sigmoidal” binding curve. The binding of proteins to nucleic acid, e.g., the lac repressor and the lac operator, are also known. The impact in the present invention is that co-operativity will detect even small quantities of target because the small molecule ligand will be in excess.

Co-operative signalling is also easily employed in the invention. The binding of one tagged ligand in proximity to another is sufficient to enhance the signal released by both tags; it is an inverse situation to signal quenching systems, which have been described above and are also well known. It may mean that the detection ligands consist of two or more different tags that can either be resident on the same ligand (like an inverse molecular beacon; see http://www.molecular-beacons.org/) or may be on separate ligands.

The invention will now be described in further detail by way of example only, with reference to the following specific embodiments.

### EXAMPLES

In one specific embodiment, virus containing body fluids, such as whole blood, plasma or serum, or urine, or cerebro-spinal fluid are reacted with a specified quantity of internally quenched detection agent.

After an incubation period, a light of specified wavelength (excitation wavelength) is shone on the reacted fluids and fluorescent emissions at a pre-determined wavelength are measured. The level of light emission is proportional to the bound fluorophore and no signal is given from unbound, and therefore quenched, fluorophore.

Although binding of single fluorescent molecule to each virus particle will give a signal, the virus particle is able to bind a multiplicity of the fluorescent molecules and thus a secondary level of signal amplification is given.

An example of molecular beacon binding to a virus particle and emissions detected from it is shown in FIG. 3. In the present embodiment, a similar plot of emission detection reveals the presence or absence of the virus particle. The intensity of the peak in the spectrum provides further quantitative information.

1.-14. (canceled)

15. A method for detecting an analyte in a sample, which method comprises:
(a) contacting the sample with a label capable of binding to the analyte;
(b) detecting the presence and/or quantity of the label bound to the analyte; and
(c) thereby detecting the presence and/or quantity of the analyte in the sample;
wherein, the analyte comprises a repeating protein unit, and wherein a plurality of label entities are capable of binding to a single analyte entity such that a signal obtained for an analyte from a plurality of labels is stronger than the signal obtained for an analyte with a single label.

16. A method according to claim 15, wherein the analyte comprises a repeating protein unit of a virus selected from adenoviruses, herpesviruses, rotaviruses, reoviruses, picomaviruses, papillomaviruses, polyomaviruses, parvoviruses, parvoviruses, influenza viruses, flaviviruses and retroviruses.

17. A method according to claim 15, wherein the virus is selected from HCV, HIV, or herpes simplex virus, human rhinovirus, influenza, or viral pathogen of non-human species.

18. (canceled)

19. A method of diagnosing the presence of a pathogen in a subject, which method comprises:
(a) obtaining a sample from the subject;
(b) detecting the absence or the presence and/or quantity of a pathogen protein in the sample according to a method as defined claim 15; and
(c) making a diagnosis of the subject based on the absence or the presence and/or quantity of the pathogen.

20. A method according to claim 19, wherein the pathogen is selected from a virus and a bacterium.

21. A method according to claim 20, wherein the virus is selected from an HCV, HIV, herpes simplex virus, human rhinovirus, influenza virus, and a viral pathogen of a non-human species.

22. A method according to claim 19 wherein the subject is a mammal.

23. A method according to claim 22 wherein the subject is human.

24. A method according to claim 15, wherein the analyte comprises a protein selected from a virus protein, a bacterial protein, and a mammalian protein.

25. A method according to claim 24, wherein the analyte is selected from a bacteriophage protein or a human protein.

26. A method according to claim 15, wherein the analyte is selected from, whole virus, viral particles, viral protein, sub-viral particles, immature viral particles, and proto-viral complexes.

27. A method according to claim 15, wherein the label comprises an antibody, an aptamer, a nucleic acid, or an organic compound or molecule.

28. A method according to claim 15, wherein the label comprises a capsid function inhibitor.

29. A method according to claim 15, wherein the label comprises an oxadiazole compound.

30. A method according to claim 29, where the label comprises Pleconaril or a Pleconaril derivative.

31. A method according to claim 15, wherein the label comprises a reporter group.

32. A method according to claim 31, wherein a property of the reporter group is altered when the label binds to the analyte.

33. A method according to claim 31, wherein the reporter group comprises a fluorescent group.

34. A method according to claim 15, wherein the signal is generated using a molecular beacon.

35. A method according to claim 15, wherein the analyte comprises HCV, HIV, or herpes simplex virus, human rhinovirus, influenza, or viral pathogen of non-human species.
